(19) United States
(12) Patent Application Publication
(10) Pub. No.: US 2024/0173269 A1
(43) Pub. Date: May 30, 2024

(54) Nitric Oxide Delivery Device for Healing Skin Wounds

(71) Applicant: NOxy Health Products, Inc., San Mateo, CA (US)

(72) Inventors: David A. Bell, Farmington, UT (US); C. Michael Miller, Anaconda, MT (US)

(21) Appl. No.: 18/506,752

(22) Filed: Nov. 10, 2023

Related U.S. Application Data
(60) Provisional application No. 63/424,221, filed on Nov. 10, 2022.

(51) Int. Cl. A61K 31/04; A61K 9/06; A61K 9/12; A61K 47/32

(52) U.S. Cl. CPC A61K 31/04; A61K 9/06; A61K 9/122; A61K 47/32

(57) ABSTRACT

This invention is directed to a topical nitric oxide formulation that can be used to treat skin conditions. The invention is also directed to a method for using the topical nitric oxide formulation to treat skin conditions, as well as to a device to administer the topical pharmaceutical formulation to an area of the skin that requires treatment.

### CROSS-REFERENCE TO RELATED APPLICATIONS

This application claims priority to U.S. Provisional Application Ser. No. 63/424,221, filed Nov. 10, 2022, the entire contents of which are incorporated by reference herein.

### FIELD OF THE INVENTION

The invention is directed to a topical pharmaceutical nitric oxide system that can be used to treat skin conditions in humans and animals. The invention is also directed to a method for using the topical nitric oxide system to treat skin conditions in humans and animals, as well as delivery devices that can be used to administer the topical pharmaceutical system to an area of the skin that requires treatment.

### BACKGROUND OF THE INVENTION

The discovery of the effect of nitric oxide on liver tissue garnered a Nobel Prize in 1992 to Rober Furchgott, Louis Ignarro, and Ferid Murad. Much of the work in determining the nitric oxide mechanism of action and possible uses of nitric oxide in medicine has since been reported in the scientific and medical literature. Today, many benefits have been documented regarding the use of nitric oxide to help or treat humans or animals having a variety of ailments, diseases, illnesses, or afflictions, particularly of the skin. Research into the therapeutic uses of nitric oxide has followed in the wake of these discoveries and continues to unveil the useful properties of nitric oxide in treating various skin pathologies that afflict humans and animals. To that end, a number of products have been developed and marketed that attempt to exploit the known benefits of nitric oxide. Numerous patents and patent applications are available that describe the benefits and uses of nitric oxide in various forms. For example, U.S. Pat. Nos. 10,751,364 and 10,543,336, disclose and claim nitric oxide compositions and delivery methods that are useful in treating skin conditions in humans and animals.

Since nitric oxide is a reactive gas with a short half-life, one of the ongoing challenges in using nitric oxide to treat skin wounds, skin diseases, and other ailments in humans and animals, is how to generate and contain the nitric oxide so that it remains in contact with skin that requires treatment. As a result, a need remains for products and methods for containing nitric oxide when it is used to treat skin conditions.

### SUMMARY OF THE INVENTION

These and other needs are met by the present invention, which is directed to a topical pharmaceutical nitric oxide system for treating skin conditions in humans and animals. The topical pharmaceutical nitric oxide system comprises nitric oxide, generated in situ, and at least one pharmaceutically acceptable excipient that is capable of forming a film or gel layer with mass transfer resistance that is suitable to contain the nitric oxide so that it remains in contact with the skin. When the topical pharmaceutical nitric oxide system is applied to an area of skin requiring treatment, the skin is covered, or “sealed” by the resultant film or gel layer, forming a barrier that blocks the escape of nitric oxide and that protects the area of skin being treated from the surrounding environment. The sealing of the wound is designed to destroy harmful bacteria and accelerate healing, by trapping moisture, oxygen, superoxide, and/or nitric oxide. Additional advantages of the system include protecting the skin requiring treatment from particles or environmental debris, air- or water-borne pathogens, as well as batch- to batch consistency, which is an important manufacturing requirement. The pharmaceutically acceptable excipient that is capable of forming a film or layer with mass transfer resistance that is suitable to contain the nitric oxide so that it remains in contact with the skin may be selected from a variety of materials known to the skilled person. For instance, the pharmaceutically acceptable excipient may be obtained from polymer coatings that are soluble in solutions that can be delivered by evaporation of solvents that do not irritate the skin.

Thus, in one aspect, what is provided is a topical nitric oxide delivery system, comprising:

a topical nitric oxide delivery system; comprising:

a first topical pharmaceutical solution comprising a nitrite, a liquid selected from the group consisting of water, one or more polar protic liquids, or a mixture of water and one or more polar liquids, and optionally one or both of a surfactant and a dermatological agent;

a second pharmaceutical solution comprising a protic acid and a liquid which is selected from the group consisting of water, one or more polar protic liquids, or a mixture of water and one or more polar protic liquids and optionally one or both of a surfactant and a dermatologic agent; wherein: a surfactant is present in at least one or both of the first and second topical pharmaceutical solution; and wherein:

at least one or both of the first and second topical pharmaceutical solutions comprise a pharmaceutically acceptable excipient or mixture of pharmaceutically acceptable excipients capable of forming a film or gelatinous layer comprised of stratified layers of nano-microbubbles containing nitric oxide when mixed.

The topical nitric oxide delivery systems disclosed herein are characterized by a film or gel that contains or is comprised of micro to nano bubbles containing nitric oxide gas. These bubbles are common throughout the entirety of the resultant stratified layers of the film or gel and help to control the directionality and release of nitric oxide to the human or animal skin that is being treated.

Also provided is a method for treating a skin condition in humans and animals, comprising contacting the skin to be treated with a topical pharmaceutical formulation that is capable of generating nitric oxide in situ. The pharmaceutical formulation comprises nitric oxide, which is generated in situ, and at least one pharmaceutically acceptable excipient. The pharmaceutical formulation containing nitric oxide is capable of forming a film or layer with greater mass transfer resistance and is able to contain the nitric oxide so that it remains in contact with the skin.

### SUMMARY OF THE FIGURES

FIG. 1A is a photographic image of the skin that formed from the topical foam formulation.

FIG. 1B is a photographic images of the skin that formed from the topical foam formulation.

FIG. 2 is a graphical depiction of NO production using a foam formulation containing 1 percent PVP by weight.

FIG. 3 is a photographic image of the skin that formed from the topical aerosol formulation.

FIG. 4 is a graphical depiction of NO production using an aerosol formulation containing 1 percent PVP by weight.

FIG. 5A is a photographic image of the skin that formed from the topical gel formulation.

FIG. 5B is a photographic image of the skin that formed from the topical gel formulation.

FIG. 6 is a graphical depiction of NO production using a gel formulation containing 5 percent PVP by weight.

### DETAILED DESCRIPTION OF THE INVENTION

### Definitions

As used herein, a skin condition is an exogenous skin wound or skin disease.

As used herein, a “skin wound” is a break or opening in the skin of a human or an animal that may require medical treatment. Non-limiting examples of skin wounds include cuts, lacerations, gashes, tears, burns, scrapes, abrasions, scratches, and bruises.

As used herein, a “skin disease” is a condition that irritates, clogs, or damages the skin of a human or animal. Non-limiting examples of skin diseases include acne, alopecia, atopic dermatitis, epidermolysis buloosa, hidradenitis suppurativa, ichythyosis, pachyonychia congenta, pemphigus, psoriasis, Reynaud's Phenomenon, rosacea, scleroderma, vitiligo, as well as itching from bug bites, poison ivy, poison oak, and the like. Skin conditions can be infected by bacteria. “Bacterial skin infection” means an infection in the skin that is caused by bacteria. Bacteria may create biofilms that can be disrupted by topical pharmaceutical agents, such as the topical nitric oxide delivery system disclosed herein. A small set of bacteria that can infect skin include P. aeruginosa, A. baumannii, S. aureus, S. epidermidis, C. albicans, S. epidermidis, P. mirabilis, Propionibacterium acnes, (P. acnes), and Malassezia spp. Bacterial skin infections include carbuncles, ecthyma, erythrasma, folliculitis, furuncles, impetigo, lymphadenitis, skin abscesses, cellulitis, erysipelas, lymphangitis, necrotizing skin infections, and wound infections.

Skin conditions can also lead to inflammation. “Inflammatory skin conditions” include a range of conditions that arise from a variety of causes. They present as skin eruptions, and rashes. Inflammatory skin conditions include eczema, psoriasis, acne, folliculitis, and allergic urticarial, atopic dermatitis, and contact dermatitis, among others.

As used herein, “dermatological agent” and “dermatological topical agent” can be used interchangeably and refer to therapeutic agents that can be applied directly on the skin of a human or an animal to treat skin conditions to deliver medicines to treat or prevent dermatologic conditions.

Types of dermatological agents include topical agents for miscellaneous use, topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacients, topical steroids, topical steroids with anti-infectives, and topical antiseptics.

Miscellaneous dermatologic agents are used to treat a variety of skin conditions. Non-limiting examples of miscellaneous topical dermatologic agents used to treat skin conditions include hydrocortisone, aluminum chloride hexahydrate, crisaborole, eflornithine, tacrolimus, pimecrolimus, minoxidil, glycopyrronium, menthol, zinc oxide, coal tar, capsaicin, selenium sulfide, bimatoprost, diphenhydramine, hydrocortisone, sodium hyaluronate, salicylic acid, Vitamin E, bexarotene, mequinol/tretinoin, becaplermin, dexpanthenol, alitretinoin, and calamine.

Topical anti-eczema agents include hydrocortisone.

Topical acne agents include antiseptic washes that contain ingredients to gently cleanse the skin; and creams, lotions, or gels that exfoliate the skin, inhibit bacterial growth, speed up skin cell renewal or decrease the formation of comedones. Non-limiting examples of topical acne agents include adapalene, benzoyl peroxide, clindamycin, dapsone, tretinoin, azelaic acid, tazarotene, salicylic acid, clascoterone, erythromycin, benzalkonium chloride, and resorcinol.

Topical anesthetics are medicines that numb and reduce the sensation of pain in the area to which they are applied. Non-limiting examples of topical anesthetics include lidocaine, pramoxine, phenol, benzocaine, dibucaine, xylocaine, tetracaine, prilocaine, and dyclonine.

Topical anti-infective agents act by either killing or inhibiting the spread of infectious agents. They include antibiotics, antibacterial, antifungal and antiviral agents. Non-limiting examples of topical anti-infectives include docosanol, ivermectin, imiquimod, hydrogen peroxide, crotamiton, spinosad, cadexomer iodine, silver, malathion, piperonyl butoxide, pyrethrins, permethrin, sinecatechins, abametapir, acetic acid, hypochlorous acid, iodoquinol, nitrofurazone, and chloroxine.

Topical anti-rosacea agents are used for the treatment of inflammatory papules, pustules and erythema of rosacea. Non-limiting examples of anti-rosacea agents include ivermectin, brimonidine, oxymetazoline, and azelaic acid.

Antibiotics are medicines that destroy or inhibit the growth of susceptible bacteria. Non-limiting examples of topical antibiotics include mupirocin, sulfacetamide sodium/sulfur, retapamulin, silver sulfadiazine, ozenoxacin, silver sulfadiazine, hypochlorous acid, erythromycin, polymyxin b, pramoxine, mafenide, gentamicin, and mupirocin.

Topical antifungals are products that treat fungal infections caused by dermatophytes, yeasts, or mold. Non-limiting examples of topical antifungals include efinaconazole, tavaborole, ketoconazole, terbinafine, undecylenic acid, ciclopirox, nystatin, econazole, ciclopirox, naftifine, oxiconazole, terbinafine, clotrimazole, sertaconazole, tolnaftate, butenafine, luliconazole, and sulconazole.

Topical antihistamines are products that have been manufactured for use on the skin, in the nose, or in the eye. They contain antihistamines which are medicines that block histamine release from histamine-1 receptors and are used to treat the symptoms of an allergic reaction such as edema (swelling), itch, inflammation (redness), sneezing, or a runny nose or watery eye. Non-limiting examples of topical antihistamines include doxepin and diphenhydramine.

Topical antineoplastics work by different mechanisms to prevent the development and spread of neoplastic cells that characterize cancers such as melanoma. Non-limiting examples of topical antineoplastics include imiquimod, fluorouracil, ingenol, ruxolitinib, tirbanibulin, and mechlorethamine.

Topical antipsoriatics are agents, which are applied on the skin surface to treat psoriasis. Non-limiting examples of topical antipsoriatics include betamethasone, calcipotriene, tazarotene, and halobetasol.

Topical antiviral agents are applied locally to treat viral infections. Non-limiting examples of topical antivirals include penciclovir and acyclovir.

Topical astringents are agents that cause skin cells or mucus membranes to contract or shrink, by precipitating proteins from their surface. When applied topically they dry, harden and protect the skin. Non-limiting examples of topical astringents are alum and witch hazel.

Debriding methods and debriding agents may also be used in combination with the topical nitric oxide delivery system disclosed herein. Debridement is the medical removal of dead, damaged, or infected tissue to improve the healing potential of the remaining healthy tissue. Removal may be surgical, mechanical, chemical, autolytic, and by other means known to the skilled person. Topical debriding agents are chemicals that are used locally to clean an open wound by removing foreign material and dead tissue, so that the wound heals without increased risk of infection. This makes the healing faster. Non-limiting examples of topical debriding agents include collagenase, balsam peru combined with castor oil and trypsin, medi-honey, and anacaulase.

Topical depigmenting agents work in different ways to inhibit melanogenesis (the pigmentation pathway by which cells produce melanin). Some agents cause reversible depigmentation and some cause irreversible depigmentation. These agents are applied on the skin, on the affected area to treat hyperpigmentation. Non-limiting examples of topical depigmenting agents include fluocinolone combined with hydroquinone and tretinoin, and monobenzoin.

Topical emollients, or moisturizers, contain ingredients that soothe and soften the skin. Non-limiting examples of topical emollients include salicylic acid/urea, ammonium lactate, urea, vitamin a, d, and e, petrolatum, lanolin, and aloe vera.

Topical keratolytics are agents that are applied on the skin to soften the keratin. They loosen and assist exfoliation of the skin cells. Keratolytics also help the skin to bind moisture and are useful in treating dry skin conditions. They are used to treat psoriasis, acne, warts, corns and other forms of keratosis. Non-limiting examples of topical keratolytincs include podofilox, salicylic acid, and trichloroacetic acid.

Topical non-steroidal anti-inflammatories (often abbreviated to NSAIDs) are creams, gels, rubs, solutions or sprays that contain a nonsteroidal anti-inflammatory agent and are designed to be applied directly to the skin overlying a painful joint or area of bone. They are used to relieve pain and to treat symptoms of arthritis such as inflammation, swelling, and stiffness. Topical NSAIDs may also be used in the treatment of actinic keratosis (a precancerous patch of thick, scaly or crusted skin). Non-limiting examples of topical NSAIDs include salicylic acid, diclofenac, capsaicin/diclofenac, and diclofenac/lidocaine.

Topical photochemotherapeutics make skin more sensitive to light. They work by causing a reaction with light that can destroy certain types of diseased skin cells. They may be used in the treatment of vitiligo or actinic keratosis in combination with light treatment. Non-limiting examples of topical photochemotherapeutics include aminolevulinic acid, methoxsalen, methylamino levulinate, and aminolevulinic acid.

Topical rubefacients cause irritation and reddening of the skin, due to increased blood flow. They are used in the treatment of pain in various musculoskeletal conditions. Non-limiting examples of topical rubefacients include methyl salicylate, camphor, menthol, phenol, trolamine salicylate, and capsaicin/menthol.

Topical steroids contain corticosteroids (often abbreviated to steroids) which are designed to be applied externally to the scalp or the skin, depending on the condition being treated. Corticosteroids control inflammation by mimicking naturally occurring corticosteroid hormones produced by adrenal glands, which are two small glands that sit on top of kidneys. In addition to reducing inflammation (redness and swelling) in the area that they are applied, topical corticosteroids also suppress the immune response, reduce cell turnover, and constrict (narrow) blood vessels. Non-limiting examples of topical steroids include mometasone, clobetasol, triamcinolone, fluocinonide, flurandrenolide, clocortolone, halobetasol, desoximetasone, desonide, hydrocortisone, betamethasone, halcinonide, fluocinolone, prednicarbate, diflorasone, fluocinolone, triamcinolone, flurandrenolide, diflorasone, fluticasone, and alclometasone.

Topical antiseptics are used to reduce the microbial count and reduce the risk of infections on the skin. Non-limiting examples of topical antiseptics include alcohol (ethanol and isopropanol), benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, and thymol. BAC represents a mixture of N,N-dimethyl alkyl amines, which conform generally to the formula:

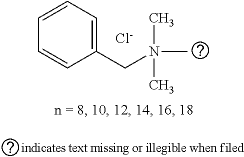

All amounts are given as weight percents based on the total weight (i.e., 100 weight percent) of a given solution in water or another solvent. Unless otherwise stated, the amount of water or other solvent is adjusted so that the total weight percent is equal to 100 weight percent.

The endpoints of ranges are to be considered as part of the range in question.

### 1. In-Situ Nitric Oxide Topical Drug Formulation

In one aspect, what is provided is a topical nitric oxide delivery system, comprising:

a topical nitric oxide delivery system; comprising:

a first topical pharmaceutical solution comprising a nitrite, a liquid selected from the group consisting of water, one or more polar protic liquids, or a mixture of water and one or more polar liquids, and optionally one or both of a surfactant and a dermatological agent;

a second pharmaceutical solution comprising a protic acid and a liquid which is selected from the group consisting of water, one or more polar protic liquids, or a mixture of water and one or more polar protic liquids and optionally one or both of a surfactant and a dermatologic agent; wherein: a surfactant is present in at least one or both of the first and second topical pharmaceutical solution; and wherein:

upon mixing, at least one or both of the first and second topical pharmaceutical solutions comprise a pharmaceutically acceptable excipient or mixture of pharmaceutically acceptable excipients capable of forming a film or gelatinous layer comprised of stratified layers of nano-microbubbles containing nitric oxide when mixed.

The topical pharmaceutical nitric oxide system for the in-situ generation of nitric oxide prepared from the mixing of first and second topical pharmaceutical solutions, wherein at least one of first and second topical pharmaceutical solutions comprises at least one film- or gel-forming pharmaceutically acceptable excipient. Because of the presence of at least one film- or gel-forming pharmaceutically acceptable excipient, the topical pharmaceutical nitric oxide system is capable of forming a film or gel layer in such a manner that the skin of a human or animal that requires treatment is covered, or sealed, by the resultant film or gel layer. The film or gel layer is comprised of micro to nano bubbles containing nitric oxide gas and these bubbles are common throughout the entirety of the resultant stratified layers of the film or gel used to seal a wound and may be a thin layer that seals the wound and thus increases the availability of nitric oxide to the area of skin requiring treatment, because the nitric oxide, once liberated from the bubbles is blocked from escaping. The wound is thus isolated in an anti-microbial environment, wherein nitric oxide inhibits bacterial infections and aids in wound healing without loss of nitric oxide to ambient interference from the environment from beyond the wound boundaries.

### A. Film- or Gel-Forming Pharmaceutically Acceptable Excipient

As noted above, the topical pharmaceutical nitric oxide system comprises a pharmaceutically acceptable film- or gel-forming layer with a mass transfer resistance that is suitable to contain the nitric oxide so that it remains in contact with the skin may be selected from a variety of materials known to the skilled person.

For instance, in one embodiment, the pharmaceutically acceptable excipient or mixture of excipients may be obtained from batch consistent polymer coatings that can be delivered from evaporation of soluble solutions in solvents that readily evaporate and that do not irritate the skin. For example, polysiloxanes such as hexamethyldisiloxane (HMDS) and isooctane may be delivered using ethanol, isopropanol, or the like. Alternatively, in another embodiment, hydrophobic alkylsiloxane-containing polymers such as those derived from the primary alkylsiloxysilane monomer 3-methacryloyloxypropyltris(trimethylsiloxy)silane (TRIS, which is also known as 3-[tris(trimethylsiloxoy)silyl]propyl methacrylate) can also be used. Such alkylsiloxane-containing polymers can be prepared in liquid polydimethylsiloxanes or isooctane. Alternatively, in another embodiment, cyanoacrylates such as n-butyl and n-octyl cyanoacrylate monomers polymerize quickly to provide films. Alternatively, in another embodiment, hydrogel materials derived from poly (N-vinyl pyrrolidone)(PVP) can also be used as the film- or gel-forming layer.

Other materials capable of achieving batch consistency such as polyurethanes, carbomers, polymethacrylate-isobutene (alcohol based), ethyl cellulose, hydroxypropyl methyl cellulose (HPMC), pyroxylin/nitrocellulose, alginates, xanthan (gum), polyols (glycols such as polyethylene glycols, and the like, may also be used.

The amount of the film- or gel-forming pharmaceutically acceptable excipient is typically in the range of 1 percent to 50 percent, or about from 1 to 25 percent, or about from 1 to 10 percent based on the total weight of the composition and can vary depending on whether the topical formulation is administered as a foam, or gel, or an aerosol.

As an alternative to, or in addition to, the addition of a gelling agent or excipient to the first or second pharmaceutical formulation, the invention also includes barrier layers that form via light or oxygen-mediated reactions. For example, cyanoacrylate tissue adhesives can be used along with the gelling topical formulation. In one embodiment, the cyanoacrylate tissue adhesive is selected from ethyl 2-cyanoacrylate (CAS Reg. No. 7085-85-0), octyl cyanoacetate (CAS Reg. No. 15666-97-4), phenytoin, or the like. The gelling topical formulation can be applied to the target wound or tissue and allowed to gel by common physical or photochemical processes, such as by heating, evaporation of solvent, or treatment with light. Cyanoacrylate tissue adhesive can then be applied on top of the gelling topical formulation as a protective second barrier that blocks the escape of nitric oxide.

In another embodiment, light activated adhesives can be implemented along with the gelling topical formulation. The gelling topical formulation can be applied to the target wound or tissue and allowed to set; i.e., to gel. The light activated adhesive can then be applied on top of the gelling topical formulation as a protective second barrier that blocks the escape of nitric oxide. For example, the adhesive can be a resin based composite or synthetic resin that can be selected from dimethacrylate monomers selected from the consisting of bisphenol A-glycidyl methacrylate (Bis-GMA), triethylene glycol (TEG) and triethylene glycol monomethacrylate (TEGMA), urethane dimethacrylate (UDMA), and hudroxyethylmethacrylate (HDDMA). The light can be a UV curing light, a tungsten-halogen curing light, or a light emitting diode curing light.

In another embodiment, heat activated adhesives can be implemented along with the gelling topical formulation. The gelling topical formulation can be applied to the target wound or tissue and allowed to set; i.e., to gel. The heat activated adhesive can then be applied on top of the gelling topical formulation as a protective second barrier that blocks the escape of nitric oxide. For example, the adhesive can be derived from poly(lactic-co-glycolic acid) and poly(ethylene glycol) (PLGA/PEG) blend fibers with or without silver salt (AgNO₃), which cure upon heating to body temperature. Daristotle et al., Bioeng Transl Med. 5, e10149, 2020 (posted at https://www.ncbi.nlm.nih.gov/pmc/articles/PMC6971445/). Alternatively, the adhesive can be a composition comprising an alginate (algae derived) hydrogel, silver nanoparticles, and Poly(N-isopropylacrylamide) (PNIPAm). Blacklow, et al., Science Advances, 5, Jul. 24, 2019 (posted at https://www.science.org/doi/10.1126/sciadv.aaw3963). The heat source can be from the body itself.

### B. First and Second Topical Pharmaceutical Solutions

The topical pharmaceutical nitric oxide system described herein can be prepared from two topical pharmaceutical solutions that, when mixed together, generate nitric oxide. At least one or both of the first pharmaceutical topical solution and second topical pharmaceutical solution comprise at least one film- or gel-forming pharmaceutically acceptable excipient that serves as a barrier or shell over the skin requiring treatment. Such gelling agents are typically hydrophilic and are known to the skilled person. As noted previously, the hydrophilic excipient can be selected from the group consisting of celluloses such as hydroxyethylcellulose, hydroxypropyl methyl cellulose (HPMC), alginates, xanthan (gum), polyols (glycols such as polyethylene glycols, polysiloxanes, polyurethanes, carbomers, pyrrolidone (PVP), and the like. In some embodiments, the gelling agent is PVP.

The film or gel layer seals the wound or area of skin requiring treatment and increases the availability of nitric oxide to the wound or area of skin requiring treatment. The sealing of the wound or area of skin requiring treatment effectively isolates the wound from the surrounding environment, so that the nitric oxide can act on bacteria and aid in wound healing directly without interference.

In one embodiment:

the first topical pharmaceutical solutions comprises:
a nitrite compound;
a polar liquid or a mixture of one or more polar liquids;
a surfactant; and

the second topical pharmaceutical solutions comprises:
a protic acid;
a liquid;
optionally a surfactant;

wherein at least one or both of the first and second topical pharmaceutical solutions comprise a pharmaceutically acceptable excipient that is capable of forming a film or layer.

In one embodiment:

the first topical pharmaceutical solutions comprises:
a nitrite compound;
a polar liquid or a mixture of one or more polar liquids;
optionally a surfactant; and

the second topical pharmaceutical solutions comprises:
a protic acid;
a liquid;
a surfactant;

wherein at least one or both of the first and second topical pharmaceutical solutions comprise a pharmaceutically acceptable excipient that is capable of forming a film or layer.

In another embodiment:

the first topical pharmaceutical solution comprises:
a nitrite compound;
a polar liquid or a mixture of one or more polar liquids;
a surfactant; and

the second topical pharmaceutical solution comprises:
a protic acid;
a liquid;
a surfactant;

wherein the first and second topical pharmaceutical solutions each comprise a pharmaceutically acceptable excipient capable of forming a film or gel. In a further embodiment, equal amounts of the pharmaceutically acceptable excipient capable of forming a film or gel are present in the first and second topical pharmaceutical solutions.

In another embodiment:

the first topical pharmaceutical solution comprises:
a nitrite compound;
a polar liquid or a mixture of one or more polar liquids;
a surfactant; and

the second topical pharmaceutical solution comprises:
a protic acid;
a liquid;
a surfactant;

wherein the first topical pharmaceutical solution comprises a pharmaceutically acceptable excipient capable of forming a film or gel.

In another embodiment:

the first topical pharmaceutical solution comprises:
a nitrite compound;
a polar liquid or a mixture of one or more polar liquids;
a surfactant; and

the second topical pharmaceutical formulation comprises:
a protic acid;
a liquid;
a surfactant;

wherein the second topical pharmaceutical solution comprises a pharmaceutically acceptable excipient capable of forming a film or gel.

As noted above, at least one or both of the first pharmaceutical topical solution and the second topical pharmaceutical solution comprise a film- or gel-forming agent. In one embodiment, the film- or gel-forming agent is selected from the group consisting of a cellulose, an alginate, a xanthan (gum), a polyol, a polysiloxane, a polyurethane, a carbomer, polyvinyl pyrrolidone, and the like, or mixtures thereof.

In another embodiment, the film- or gel-forming agent is selected from the group consisting of hydroxyethylcellulose, hydroxypropyl methyl cellulose (HPMC), and polyvinyl pyrrolidone (PVP), or mixtures thereof In one embodiment, the gelling agent is PVP.

In one embodiment of the topical nitric oxide delivery system described herein, the first pharmaceutical solution comprises a pharmaceutically acceptable excipient or mixture of excipients capable of forming a film or gelatinous layer.

In another embodiment of the topical nitric oxide delivery system described herein, the second pharmaceutical solution comprises a pharmaceutically acceptable excipient or mixture of excipients capable of forming a film or gelatinous layer.

In another embodiment of the topical nitric oxide delivery system described herein, the first pharmaceutical solution and the second pharmaceutical solution each comprise the same or different pharmaceutically acceptable excipient or mixture of excipients capable of forming a film or gelatinous layer.

In one embodiment of the first topical pharmaceutical solution, the nitric oxide is generated from a nitrite derived from a Group I or Group II element of the Periodic Table of the Elements. In one embodiment, the nitric oxide precursor is selected from the group consisting of sodium nitrite, potassium nitrite, calcium nitrite, and magnesium nitrite. In one embodiment, the nitric oxide precursor is sodium nitrite.

In this and other embodiments of the first topical pharmaceutical solution, the liquid is water, or water mixed with one or more other polar liquids. The one or more polar liquids can be selected based on a physical parameter such as boiling point or vapor pressure, to adjust the time to gel formation. In this and other embodiments, the liquid is selected from the group consisting of an alcohol, an ether, a ketone, an ester, or any other pharmaceutically acceptable polar liquid. In a further embodiment, the polar liquid is selected from the group of ethanol, isopropyl alcohol, methyl ethyl ketone, ethyl acetate, ethylene glycol, propylene glycol, butylene glycol, glycerin, polysorbates such TWEEN 20, TWEEN 40, and TWEEN 80, and cyclodextrins (such as hydroxypropyl-(3-cyclodextrin), polyalkylene glycols, such as polyethylene glycol, polypropylene glycol, and polybutylene glycol, and primary amides such as niacinamide, or mixtures thereof.

In this and other embodiments of the first topical pharmaceutical solution, the surfactant is a pharmaceutically acceptable surfactant that can be used topically on the skin and that does not react with the other components. In a further embodiment, the surfactant is selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride; or combinations thereof. In one embodiment, the surfactant is coco betaine.

In one embodiment of the second topical pharmaceutical solution, the protic acid is an acid having a pKa of 2-6 to provide a solution that has a pH of approximately 2 to 6.9. Thus, the protic acid is selected form the group consisting of the common mineral acids as well as Aloe vera gel, lidocaine, ascorbic acid, citric acid, honey, and salicylic acid. In this and other embodiments, as with the first pharmaceutical liquid, the liquid of the second topical pharmaceutical solution can be one or more polar liquids can be selected based on a physical parameter such as boiling point or vapor pressure, to adjust the time to gel formation. In this and other embodiments, the liquid is selected from the group consisting of an alcohol, an ether, a ketone, an ester, or any other pharmaceutically acceptable polar liquid. In a further embodiment, the polar liquid is selected from the group of ethanol, isopropyl alcohol, methyl ethyl ketone, ethyl acetate, ethylene glycol, propylene glycol, butylene glycol, glycerin, polysorbates such TWEEN 20, TWEEN 40, and TWEEN 80, and cyclodextrins (such as hydroxypropyl-(3-cyclodextrin), polyalkylene glycols, such as polyethylene glycol, polypropylene glycol, and polybutylene glycol, and primary amides such as niacinamide, or mixtures thereof.

In this and other embodiments of the second topical pharmaceutical solution, the optional surfactant is a pharmaceutically acceptable surfactant that can be used topically on the skin and that does not react with the other components. In a further embodiment, the surfactant is selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride; or combinations thereof. In one embodiment, the surfactant is coco betaine.

In a further embodiment, a first topical pharmaceutical solution may comprise approximately 100 mL of water, 10 g of sodium nitrite, and 1 g of the cationic surfactant. A second topical pharmaceutical formulation may comprise approximately 100 mL of water, 5 g of lactic acid, 6 g of citric acid, and 3 g of the cationic surfactant. The film- or gel-forming pharmaceutically acceptable excipient which may be part of the first or second topical pharmaceutical formulations, or both, and is typically present in an amount of 1 to 50 6, 1 to 25 g, or 1 to 10 g per 100 mL of water.

In another embodiment, a first topical pharmaceutical solution may comprise approximately 100 mL of water alone or as a mixture with a polar protic liquid, 10 g of sodium nitrite, 2 g of sodium bicarbonate, and 1 g of the cationic surfactant. A second topical pharmaceutical solution may comprise approximately 100 mL of water alone or as a mixture with a polar protic liquid, 5 g of lactic acid, 8 g of citric acid, and 3 g of the cationic surfactant. The hydrophilic pharmaceutically acceptable excipient that is capable of forming a film or a gel may be part of the first or second topical pharmaceutical solutions, or both, and is typically present in an amount of 1 to 50 6, 1 to 25 g, or 1 to 10 g per 100 mL of water.

In another embodiment, a first topical pharmaceutical solution may comprise approximately 100 mL of water alone or as a mixture with a polar protic liquid, 10 g of sodium nitrite, and 2 g of the cationic surfactant. A second topical pharmaceutical solution may comprise approximately 100 mL of water alone or as a mixture with a polar protic liquid, 5 g of lactic acid, 6 g of citric acid, and 6 g of the cationic surfactant. The hydrophilic pharmaceutically acceptable excipient that is capable of forming a film or a gel may be part of the first or second topical pharmaceutical solutions, or both, and is typically present in an amount of 1 to 50 6, 1 to 25 g, or 1 to 10 g per 100 mL of water.

In a further embodiment, a first topical pharmaceutical solution may comprise approximately 100 mL of water alone or as a mixture with a polar protic liquid, 10 g of sodium nitrite, 2 g of sodium bicarbonate, and 1 g of the cationic surfactant. A second topical pharmaceutical solution may comprise approximately 100 mL of water alone or as a mixture with a polar protic liquid, 5 g of lactic acid, 8 g of citric acid, and 6 g of the cationic surfactant. The hydrophilic pharmaceutically acceptable excipient that is capable of forming a film or a gel may be part of the first or second topical pharmaceutical solutions, or both, and is typically present in an amount of 1 to 50 6, 1 to 25 g, or 1 to 10 g per 100 mL of water.

In one embodiment, a first topical pharmaceutical solution may comprise approximately 100 mL of water, 10 g of sodium nitrite, and 2 g of a cationic surfactant. A second topical pharmaceutical solution may comprise approximately 100 mL of water, 5 g of lactic acid, 6 g of citric acid, and 6 g of the cationic surfactant. The hydrophilic pharmaceutically acceptable excipient that is capable of forming a film or a gel may be part of the first or second topical pharmaceutical formulations, or both. A mixture of a first portion of the first topical pharmaceutical formulation and a second portion of the second topical pharmaceutical formulation may be mixed, thereby producing micro- to nano-bubbles containing nitric oxide gas upon the mixing of the first and second portions.

In additional embodiments, the first solution may further comprise a salt buffer that regulates the reaction rate of nitric oxide production. Such buffers are well known to the skilled person.

In further embodiments, the topical pharmaceutical formulation can comprise an additional therapeutic agent suitable for topical formulation as a component of the first pharmaceutical solution or the second pharmaceutical solution, or both. As used herein, “dermatological agent” and “dermatological topical agent” can be used interchangeably and refer to therapeutic agents that can be applied directly on the skin to treat skin conditions as described herein. They deliver medicines to treat skin conditions such as wounds or dermatologic diseases. Non-limiting examples of such dermatologic diseases and conditions are provided in the definitions section of this application.

In further embodiments, at least one of the solutions of the topical dermatologic solution further comprises a topical dermatologic agent or a combination of dermatologic agents. In another embodiment, both solutions comprise a topical dermatologic agent or combination of agents. In another embodiment, the first solution comprises a topical dermatologic agent, and the second solution comprises a different topical dermatologic agent.

In these and other embodiments, the topical dermatologic agent is selected from the group of agents provided in the definitions section of this application, consisting of topical acne agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical, depigmenting agents, topical emollients, topical keratolytics, topical non-steroidal anti-inflammatories, topical photochemotherapeutics, topical rubefacients, topical steroids, topical steroids with anti-infectives, antieczema agents, and topical antiseptics, or a combination thereof. Non-limiting examples of various topical dermatologic agents are provided in the definitions section of this application.

In another embodiment, the topical dermatologic formulation disclosed herein comprises a topical dermatologic antiseptic for first aid use to treat wounds including scrapes, cuts and burns. In this and other embodiments, the topical dermatologic antiseptic is selected from the group consisting of ethanol, isopropanol, benzethonium chloride, benzalkonium chloride (BAC), camphorated metacresol, eucalyptol 0.091%, hexylresorcinol hydrogen peroxide topical solution, iodine tincture, iodine topical solution, menthol, methylbenzethonium chloride, methyl salicylate, phenol, povidone-iodine, and thymol.

In these and other embodiments, the topical dermatologic agent is present based on the total weight of either the first solution, second solution, or both the first and second solutions, of 0.01 to 5 weight percent.

### C. Resultant Topical Pharmaceutical Formulation

As disclosed herein, when a portion of the first and second solutions are mixed, a resultant mixed medium is produced that contains nitric oxide. The solutions are mixed, typically again manually, to produce a carrier for the in situ generation of nitric oxide due to the reaction of sodium nitrite or potassium nitrite with acid, according to the reaction sequence shown below.

NO₂⁻+H⁺↔HNO₂(pKa3.2 to 3.4) (1)

2HNO₂→N₂O₃+H₂O (2)

N₂O₂→NO+NO₂ (3)

In one embodiment, the resultant mixed medium is a foam, gel, aerosol, or other medium that is readily applied to the skin.

As indicated previously, the gelling agent may be a component of one or both of the first or second pharmaceutically acceptable topical formulations. Alternatively, the gelling agent may be provided by an additional, separate pharmaceutically acceptable medium. For example, and not by way of limitation, a gel or serum may dry and form a barrier or shell that keeps nitric oxide gas liberated from the rupture of the nitric oxide gas containing micro-nanobubbles from escaping, creating a covered, wound-healing, anti-microbial environment or domain promoting healing of the wound. The barrier over the wound may be air-tight. More than one barrier, and/or more than one type of barrier, may be used. The barrier over the wound may be comprised of a polar compound or component such as a hydrophilic excipient as described previously. In another embodiment:

the first topical pharmaceutical solution is in water or water-alcohol and comprises a nitric oxide donor selected from the group consisting of sodium nitrite and potassium nitrite or a mixture thereof, and a surfactant selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride; or combinations thereof;

the second topical pharmaceutical solution is in water or water-alcohol and comprises and acid selected from the group consisting of citric acid, lactic acid, and ascorbic acid or a mixture thereof, and a surfactant selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride; or combinations thereof; and

the pharmaceutically acceptable excipient that is capable of forming a film or gelatinous layer is selected from the group consisting of a cellulose, an alginate, a xanthan (gum), a polyol, a polysiloxane, a polyurethane, a carbomer, and polyvinyl pyrrolidone, or mixtures thereof;

wherein the pharmaceutically acceptable excipient that is capable of forming a film or gelatinous layer is present in at least one or both of the first and second pharmaceutical solutions. In another embodiment:

the first topical pharmaceutical solution is in water or water-alcohol and comprises a nitric oxide donor selected from the group consisting of sodium nitrite and potassium nitrite or a mixture thereof and coco betaine;

the second topical pharmaceutical solution is in water or water-alcohol and comprises an acid selected from the group consisting of citric acid, lactic acid, and ascorbic acid, or a mixture thereof and coco betaine; and

PVP;

wherein the PVP is present in at least one or both of the first and second pharmaceutical solutions.

In another embodiment of the previous embodiments, the liquid is water or water-mixed with alcohol, wherein the alcohol is selected from the group consisting of ethanol and isopropanol.

In another embodiment, the liquid is water-ethanol.

Further embodiments are provided in the following Tables 1A-1C.

TABLE 1A

| Topical Pharmaceutical Solution B | Topical Solution A |
|---|---|
| (percent by weight in water) | (percent by weight in water) |
| 1 to 35 percent nitrite | 1 to 20 percent protic acid |
| 0.1 to 10 percent surfactant | 0.1 to 10 percent surfactant |
| 1 to 50 percent film or gel-forming | |
| excipient | |

TABLE 1B

| Topical Pharmaceutical Solution B | Topical Pharmaceutical Solution A |
|---|---|
| (percent by weight in water) | (percent by weight in water) |
| 1 to 35 percent nitrite | 1 to 20 percent protic acid |
| 0.1 to 10 percent surfactant | 0.1 to 10 percent surfactant |
| | 1 to 50 percent film or gel-forming |
| | excipient |

TABLE 1C

| Topical Pharmaceutical Solution B | Topical Pharmaceutical Solution A |
|---|---|
| (percent by weight in water) | (percent by weight in water) |
| 1 to 35 percent nitrite | 1 to 20 percent protic acid |
| 0.1 to 10 percent surfactant | 0.1 to 10 percent surfactant |
| 1 to 50 percent film or gel-forming | 1 to 50 percent film or gel-forming |
| excipient | excipient |

Further embodiments are provided in the following Tables 2A-2C.

TABLE 2A

| Topical Pharmaceutical Solution B | Topical Pharmaceutical Solution A |
|---|---|
| (percent by weight in water) | (percent by weight in water) |
| 5 to 25 percent nitrite | 3 to 18 percent protic acid |
| 0.3 to 8 percent surfactant | 0.5 to 8 percent surfactant |
| 1 to 25 percent film or gel-forming | |
| excipient | |

TABLE 2B

| Topical Pharmaceutical Solution B | Topical Pharmaceutical Solution A |
|---|---|
| (percent by weight in water) | (percent by weight in water) |
| 5 to 25 percent nitrite | 3 to 18 percent protic acid |
| 0.3 to 8 percent surfactant | 0.5 to 8 percent surfactant |
| | 1 to 25 percent film or gel-forming |
| | excipient |

TABLE 2C

| Topical Pharmaceutical Solution B | Topical Pharmaceutical Solution A |
|---|---|
| (percent by weight in water) | (percent by weight in water) |
| 5 to 25 percent nitrite | 3 to 18 percent protic acid |
| 0.3 to 8 percent surfactant | 0.5 to 8 percent surfactant |
| 1 to 25 percent film or gel-forming | 1 to 25 percent film or gel-forming |
| excipient | excipient |

Further embodiments are provided in the following Tables 3A to 3C.

TABLE 3A

| Topical Pharmaceutical Solution B | Topical Pharmaceutical Solution A |
|---|---|
| (percent by weight in water) | (percent by weight in water) |
| 10 to 20 percent nitrite | 5 to 15 percent protic acid |
| 0.5 to 5 percent surfactant | 1 to 5 percent surfactant |
| 1 to 10 percent film or gel-forming | |
| excipient | |

TABLE 3B

| Topical Pharmaceutical Solution B | Topical Pharmaceutical Solution A |
|---|---|
| (percent by weight in water) | (percent by weight in water) |
| 10 to 20 percent nitrite | 5 to 15 percent protic acid |
| 0.5 to 5 percent surfactant | 1 to 5 percent surfactant |
| | 1 to 10 percent film or gel-forming |
| | excipient |

TABLE 3C

| Topical Pharmaceutical Solution B | Topical Pharmaceutical Solution A |
|---|---|
| (percent by weight in water) | (percent by weight in water) |
| 10 to 20 percent nitrite | 5 to 15 percent protic acid |
| 0.5 to 5 percent Coco betaine | 1 to 5 percent surfactant |
| 1 to 10 percent PVP | 1 to 10 percent film or gel-forming |
| | excipient |

In these and other embodiments, water as the solvent can be replaced with water alcohol, such as water-ethanol or water isopropanol. In further embodiments, the solvent is 50 percent water/50 percent ethanol.

Further embodiments are provided in the following Tables 4A to 4C.

TABLE 4A

| Topical Pharmaceutical Solution B | Topical Solution A |
|---|---|
| (percent by weight in water or | (percent by weight in water or |
| water-ethanol) | water-ethanol) |
| 1 to 35 percent sodium nitrite | 1 to 20 percent citric acid |
| 0.1 to 10 percent Coco betaine | 0.1 to 10 percent Coco-betaine |
| 1 to 50 percent PVP | |

TABLE 4B

| Topical Pharmaceutical Solution B | Topical Pharmaceutical Solution A |
|---|---|
| (percent by weight in water or | (percent by weight in water or |
| water-ethanol) | water-ethanol) |
| 1 to 35 percent sodium nitrite | 1 to 20 percent citric acid |
| 0.1 to 10 percent Coco betaine | 0.1 to 10 percent surfactant |
| | 1 to 50 percent PVP |

TABLE 4C

| Topical Pharmaceutical Solution B | Topical Pharmaceutical Solution A |
|---|---|
| (percent by weight in water or | (percent by weight in water or |
| water-ethanol) | water-ethanol) |
| 1 to 35 percent sodium nitrite | 1 to 20 percent citric acid |
| 0.1 to 10 percent Coco betaine | 0.1 to 10 percent Coco betaine |
| 1 to 50 percent PVP | 1 to 50 percent film or gel-forming |
| | excipient |

Further embodiments are provided in the following Tables 5A-5C.

TABLE 5A

| Topical Pharmaceutical Solution B | Topical Pharmaceutical Solution A |
|---|---|
| (percent by weight in water or | (percent by weight in water or |
| water-ethanol) | water-ethanol) |
| 5 to 25 percent sodium nitrite | 3 to 18 percent citric acid |
| 0.3 to 8 percent Coco betaine | 0.5 to 8 percent Coco betaine |
| 1 to 25 percent PVP | |

TABLE 5B

| Topical Pharmaceutical Solution B | Topical Solution A |
|---|---|
| (percent by weight in water or | (percent by weight in water or |
| water-ethanol) | water-ethanol) |
| 5 to 25 percent sodium nitrite | 3 to 18 percent citric acid |
| 0.3 to 8 percent Coco betaine | 0.5 to 8 percent Coco betaine |
| | 1 to 25 percent PVP |

TABLE 5C

| Topical Pharmaceutical Solution B | Topical Solution Formulation A |
|---|---|
| (percent by weight in water or | (percent by weight in water or |
| water-ethanol) | water-ethanol) |
| 5 to 25 percent sodium nitrite | 3 to 18 percent citric acid |
| 0.3 to 8 percent Coco betaine | 0.5 to 8 percent Coco betaine |
| 1 to 25 percent PVP | 1 to 25 percent PVP |

Further embodiments are provided in the following Tables 6A to 6C.

TABLE 6A

| Topical Pharmaceutical Solution B | Topical Solution A |
|---|---|
| (percent by weight in water or | (percent by weight in water or |
| water-ethanol) | water-ethanol) |
| 10 to 20 percent sodium nitrite | 5 to 15 percent citric acid |
| 0.5 to 5 percent Coco betaine | 1 to 5 percent Coco betaine |
| 1 to 10 percent PVP | |

TABLE 6B

| Topical Pharmaceutical Solution B | Topical Pharmaceutical Solution A |
|---|---|
| (percent by weight in water or | (percent by weight in water or |
| water-ethanol) | water-ethanol) |
| 10 to 20 percent sodium nitrite | 5 to 15 percent citric acid |
| 0.5 to 5 percent Coco betaine | 1 to 5 percent Coco betaine |
| | 1 to 10 percent PVP |

TABLE 6C

| Topical Pharmaceutical Solution B | Topical Pharmaceutical Solution A |
|---|---|
| (percent by weight in water or | (percent by weight in water or |
| water-ethanol) | water-ethanol) |
| 10 to 20 percent sodium nitrite | 5 to 15 percent citric acid |
| 0.5 to 5 percent Coco betaine | 1 to 5 percent Coco betaine |
| 1 to 10 percent PVP | 1 to 10 percent PVP |

In a further embodiment, the formulation is a foam as described in Tables 7A or 7B.

TABLE 7A

| | Mass/Mass Percent |
|---|---|
| | Based on 100 Percent |
| Foam Solution B | |
| Acid | 1 to 18 |
| PVP | 1 to 10 |
| DI Water | Remainder |
| Surfactant | 1 to 20 |
| Foam Solution A | |
| Sodium Nitrite | 1 to 30 |
| PVP | 1 to 10 |
| DI Water | Remainder |
| Surfactant | 1 to 20 |

TABLE 7B

| | Mass/Mass Percent |
|---|---|
| | Based on 100 Percent |
| Foam Solution B | |
| Acid | 8 to 12 |
| PVP | 1 to 4 |
| DI Water | Remainder |
| Surfactant | 8 to 12 |
| Foam Solution A | |
| Sodium Nitrite | 14 to 18 |
| PVP | 1 to 5 |
| DI Water | Remainder |
| surfactant | 1 to 20 |

In a further embodiment, the formulation is an aerosol as described in Tables 8A or 8B.

TABLE 8A

| | Mass/Mass Percent |
|---|---|
| | Based on 100 Percent |
| Aerosol Solution B | |
| Acid | 1 to 18 |
| PVP | 1 to 10 |
| DI Water | Remainder |
| Aerosol Solution A | |
| Sodium Nitrite | 1 to 30 |
| PVP | 1 to 10 |
| DI Water | Remainder |

TABLE 8B

| | Mass/Mass Percent |
|---|---|
| | Based on 100 Percent |
| Aerosol Solution B | |
| Acid | 8 to 12 |
| PVP | 1 to 5 |
| DI Water | Remainder |
| Aerosol Solution A | |
| Sodium Nitrite | 14 to 18 |
| PVP | 1 to 5 |
| DI Water | Remainder |

In a further embodiment, the formulation is a gel as described in Tables 9A or 9B.

TABLE 9A

| | Mass/Mass Percent |
|---|---|
| Gel Solution B | |
| Acid | 1 to 18 |
| PVP | 1 to 30 |
| DI Water | Remainder |
| Alcohol | 5 to 95 |
| Gel Solution A | |
| Sodium Nitrite | 1 to 30 |
| PVP | 1 to 30 |
| DI Water | Remainder |
| Ethanol | 5 to 95 |

TABLE 9B

| | Mass/Mass Percent |
|---|---|
| Gel Solution B | |
| Acid | 8 to 12 |
| PVP | 1 to 10 |
| DI Water | Remainder |
| Alcohol | 40 to 50 |
| Gel Solution A | |
| Sodium Nitrite | 14 to 18 |
| PVP | 1 to 10 |
| DI Water | Remainder |
| Alcohol | 40 to 50 |

In accordance with the foregoing aspect and embodiments, other aspects and embodiments are directed to products, compounds, and methods that are employed to provide a topical nitric oxide formulation that can be applied to the skin of a human or animal that requires treatment to promote the production of nitric oxide to treat various skin conditions as described herein. The topical nitric oxide formulation may be suitably formulated to promote production of nitric oxide for use in human and animal cells and tissues.

In one embodiment, the first and second topical pharmaceutical solutions may be contained in separate containers. For example, the first topical pharmaceutical solution may be contained in a first container and the second topical pharmaceutical solution may be contained in a second container. Each container may then dispense the respective topical pharmaceutical formulation as a medium, for instance, a gel, an emulsion, a suspension, a foam or foamy suspension, an aerosol, or any other dispensation.

In one embodiment, the topical pharmaceutical formulation can be administered as a foam that is generated upon mixing of the first and second topical pharmaceutical formulations described previously. In one embodiment, the first topical pharmaceutical formulation comprises sodium nitrite and water, and the second topical pharmaceutical formulation comprises water and an acid such as citric acid. In this and other embodiments the first and second topical pharmaceutical formulations may further comprise a surfactant.

The foamy suspension can be mixed prior to application on the skin to be treated or mixed on the skin to be treated. Any suitable container may be used to contain the first and second topical pharmaceutical formulations. In certain embodiments, a container that is capable of aerating and dispensing the solution as a foam may be used. For example, dispensers like those described in U.S. Patent Application US20130200109 and U.S. Pat. No. 7,066,356 may be utilized. Also, a dispenser like that sold under the tradename DIAL® Complete foaming anti-bacterial hand soap may also be utilized.

In a further embodiment, the first topical pharmaceutical solution may comprise water, sodium nitrite, and optionally a cationic surfactant. A suitable cationic surfactant may include coco-betaine, or cocamidopropyl betaine. The second topical pharmaceutical solution may comprise water, lactic acid, citric acid, and optionally a cationic surfactant such as coco-betaine, or cocamidopropyl betaine. The hydrophilic pharmaceutically acceptable excipient that is capable of forming a film or a gel may be part of the first or second topical pharmaceutical formulations, or both.

A portion of the first topical pharmaceutical solution may be dispensed from the first container in a manner that produces a first foam. A portion of the second topical pharmaceutical solution may then be dispensed from the second container in a manner that produces a second foam. The first and second foams may be mixed to produce a resultant foam. Upon mixing of the first and second foams, nitric oxide generation begins. The resultant foam may be applied to a portion of skin of a human or animal intended to be treated. While the foam collapses, a film or gelatinous layer may form to cover the skin, thus sealing in remaining nitric oxide and blocking exposure of the skin that requires treatment to the surrounding environment. The resultant formulation may be allowed to remain on the portion of skin for a longer amount of time. After an appropriate period of time, the formulation may be removed from the skin by peeling or by rinsing, wiping, or the like.

In one embodiment, the first and second topical pharmaceutical solutions are mixed and agitated to produce foams, and then the two foams are combined to form the resultant medium. Typically the first and second pharmaceutical solutions are available in first and second plastic squirt bottles that can be shaken by hand to produce foams or mechanically pumped or otherwise forced through screens to produce foams. The two foams are mixed manually or by persuading the foam to transit an inline mixer to produce a foam carrier for the in situ generation of nitric oxide. The foam is then applied to the skin of a human or animal in need of treatment. Evaporation of the liquid from the mixture leaves a gelatinous residue on the skin being treated that covers, or seals, the skin from the outside environment.

In another embodiment, the first and second topical pharmaceutical solutions are mixed to form a medium. Typically, the first and second topical pharmaceutical solutions are available in first and second plastic squirt bottles that can be mechanically pumped and mixed, typically again manually, to produce a medium for the in situ generation of nitric oxide in micro-nanobubbles containing nitric oxide that is capable of gelling. The medium is then applied to the skin of a human or animal in need of treatment. Evaporation of the liquid from the mixture leaves a gelatinous residue on the skin being treated that covers, or seals, the skin from the outside environment.

In an alternative gel embodiment, the water-soluble protic liquid mixture, wherein the vapor pressure of the water soluble protic liquid is higher than water.

In a further gel embodiment, mineral oil and glycerol can replace water as the solvent.

In another embodiment, the first and second topical pharmaceutical solutions are mixed to form an aerosol. Typically, the first and second topical pharmaceutical solutions are available in first and second plastic squirt bottles that can be mechanically pumped and mixed, to produce an aerosol medium for the in situ generation of nitric oxide that is capable of gelling. The aerosol is then applied to the skin of a human or animal in need of treatment. Evaporation of the liquid from the mixture leaves a gelatinous residue on the skin being treated that covers, or seals, the skin from outside environment.

As noted herein, the topical pharmaceutical nitric oxide system of the present invention can be used to treat skin conditions of animals and humans.

Thus, in one aspect, what is provided is a method for providing nitric oxide therapy to the skin of a human or animal requiring treatment, comprising:

providing a first topical pharmaceutical solution comprising a nitrite compound, a surfactant, a liquid selected from the group consisting of water, one or more polar protic liquids, or a mixture of water and one or more polar protic liquids;

providing a second pharmaceutical solution comprising a protic acid and a liquid which is selected form the group consisting of water, one or more polar protic liquids, or a mixture of water and one or more polar protic liquids, wherein at least one or both of the first and second topical pharmaceutical solutions comprise a pharmaceutically acceptable excipient that is capable of forming a film or gelatinous layer;

dispensing the first and second topical pharmaceutical solutions;

mixing the first and second topical pharmaceutical formulations to initiate the production of nitric oxide containing micro-nanobubbles within the mixture;

applying the mixture to the skin of the human or animal requiring treatment; and

allowing the mixture to form a barrier enclosing the skin of the human or animal requiring treatment and trapping at least a portion of the foam between the barrier and the wound.

In one embodiment of this aspect, dispensing can be any method of dispensation suitable for a particular medium that is known to the skilled person. For a foam, dispensation can be by a pump squirt from a bottle equipped with a pump. For a gel, dispensation can be by pouring, pumping, scooping, or spreading, or the like, depending on the physical characteristics of the gel. For an aerosol, dispensation can be by pumping or spraying.

In another embodiment, mixing can be any method of mixing that is known to the skilled person. For example, mixing can be in a sterile vessel such as a petri dish prior to application. Alternatively, mixing can be achieved by virtue of a pump mechanism into a mixing valve or compartment. Alternatively, for aerosols, mixing can be achieved by virtue of mixing of two aerosol streams in air as they are sprayed.

In another aspect, what is provided is a method of treating a skin condition of a human or animal, comprising:

combining the first and second topical pharmaceutical formulations as provided herein to generate a mixture containing stratified layers of micro- to nano-bubbles containing nitric oxide;

applying the combination to the skin that requires treatment; and

allowing the mixture to form a barrier enclosing the skin that requires treatment and trapping at least a portion of the foam between the barrier and the wound.

In a further aspect, what is provided is a method of treating a skin condition of a human or animal, comprising

combining the first and second topical pharmaceutical formulations recited in claims 1-9 to generate a mixture containing stratified layers of micro- to nano-bubbles containing nitric oxide on the skin requiring treatment to form a mixture; and allowing the mixture to form a barrier enclosing the requiring treatment and trapping at least a portion of the foam between the barrier and the wound.

The invention will now be demonstrated by the following non-limiting examples.

### EXAMPLE

### Introduction

We tested in-situ nitric oxide foam, gel, and aerosol formulations containing a thickening agent to see if the formulations form a skin when applied to a testing surface (the bottom of a weigh boat). Foam, gel, and aerosol formulations were created and the rate of NO production was measured. Each example formulation produced NO and left behind a skin residue upon drying. In each case, a film started forming as soon as the foam, gel or aerosol was applied to the testing surface. After 18-24 hours of drying, formation of a skin was confirmed.

### Test Methods and Results

### A. Foam Formulation

Solutions A and B containing 1 percent PVP by weight were each prepared in pump bottles as provided in Table 10.

TABLE 10

| | Mass/Mass Percent |
|---|---|
| | Based on 100 Percent |
| Foam Solution B | |
| Acid | 1 to 18 |
| PVP | 1 to 10 |
| DI Water | Remainder |
| Surfactant | 1 to 20 |
| Foam Solution A | |
| Sodium Nitrite | 1-30 |
| PVP | 1 to 10 |
| DI Water | Remainder |
| surfactant | 1 to 20 |

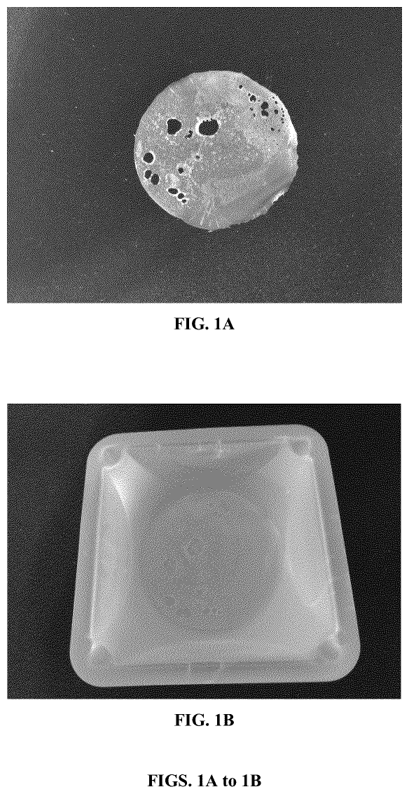

The amount of PVP was enough to form a skin (see FIGS. 1A and 1B), but not so much that the viscosity of the A and B solutions increased to the point where the foamer pump no longer worked.

### 1. No Production

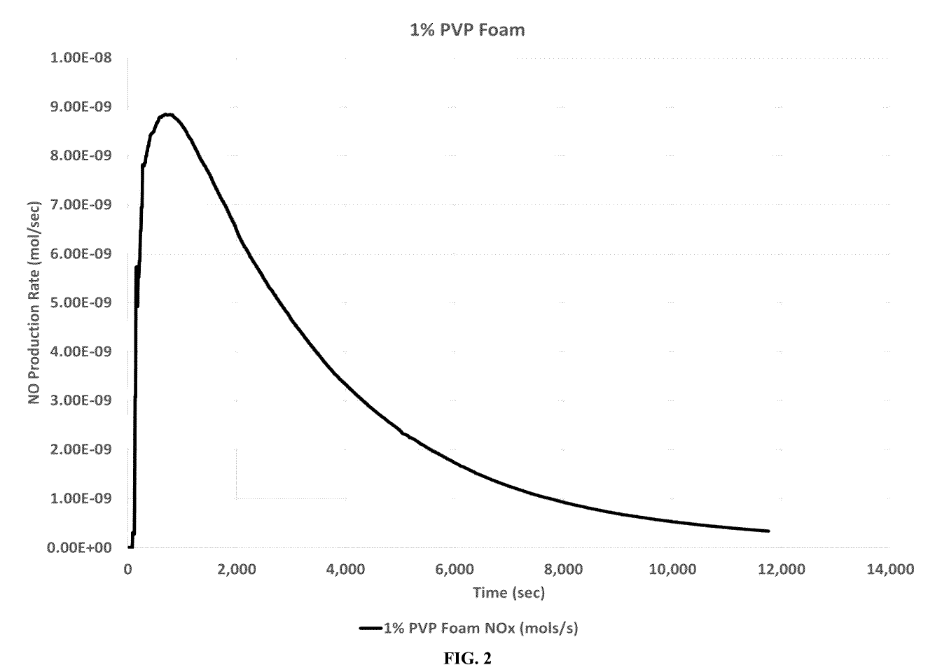

NO production for the foam formulation was measured using a Thermo Scientific™ 42iQLS Low Source NO-NO2-NOx Analyzer. The Analyzer was warmed up for 30 minutes before testing commenced. The data logging program was started with the filename YYMMDD [Solution] Test [number]. Solution A and Solution B were shaken until they each formed a foam. One pump of Solution A foam and one pump of Solution B foam were placed in a weigh boat. The contents of the weigh boat were then mixed for 5 seconds manually. The weigh boat was then placed into the sampling bin of the 42iQLS analyzer, and the lid placed on top. The temperature of the pad was set to 37° C. prior to placing the weigh boat in the chamber. The 42iQLS analyzer began measuring NOₓ released from the mixed foam in the weigh boat as indicated by the rise of both the NO and NO₂ measurements on the screen of the analyzer. Data collection stopped after 700 seconds of data, which provided 70 individual time points. NO production is shown in FIG. 2.

### B. Aerosol Formulation

Solutions A and B containing 1 percent PVP and 5 percent PVP by weight, respectively, were prepared in aerosol pump bottles according to Table 11.

TABLE 11

| | Mass/Mass Percent |
|---|---|
| | Based on 100 Percent |
| Aerosol Solution B | |
| Acid | 1 to 18 |
| PVP | 1 to 10 |
| DI Water | Remainder |
| Aerosol Solution A | |
| Sodium Nitrite | 1 to 30 |
| PVP | 1 to 10 |
| DI Water | Remainder |

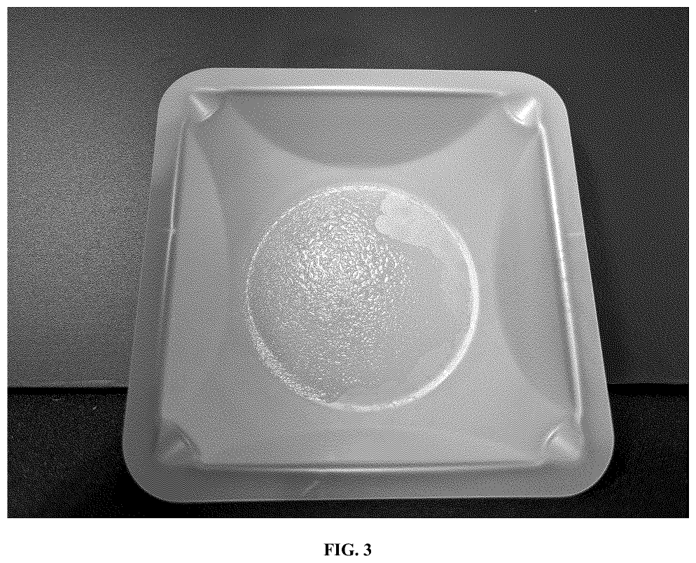

The amount of PVP was enough to form a skin (see FIG. 3), but not so much that the viscosity of the A and B solutions was increased to the point where the aerosol pumps no longer worked.

### 1. No Production

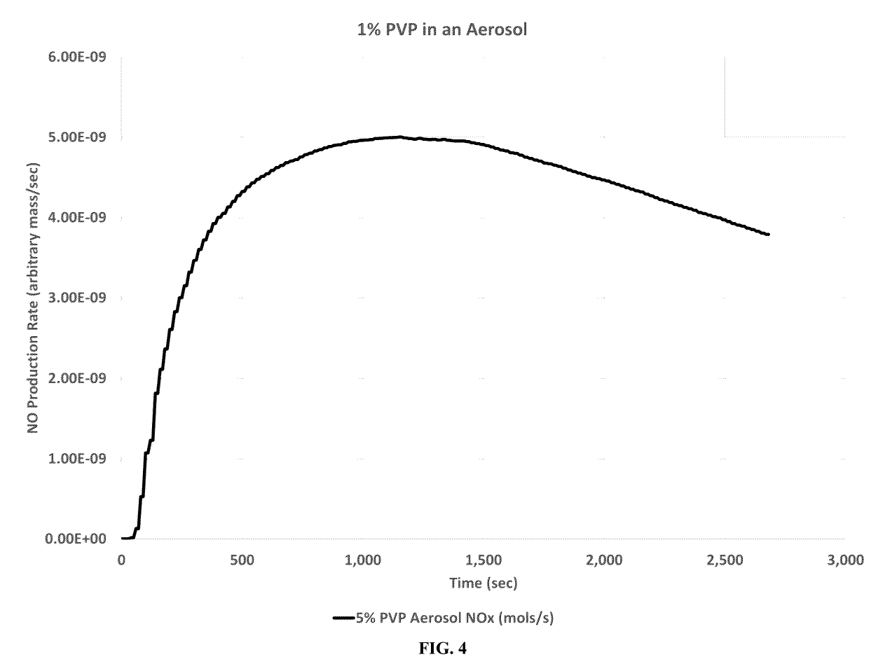

For each formulation, one aerosol pump of aerosol A and one pump of aerosol B were combined in a weigh boat. The NO production rate was measured as described in Part A with the temperature of the pad set to 37° C. The NO production profiles for both formulations are shown in FIG. 4. The vertical axis of the graph is in arbitrary mass units per second.

### C. Gel Formulation

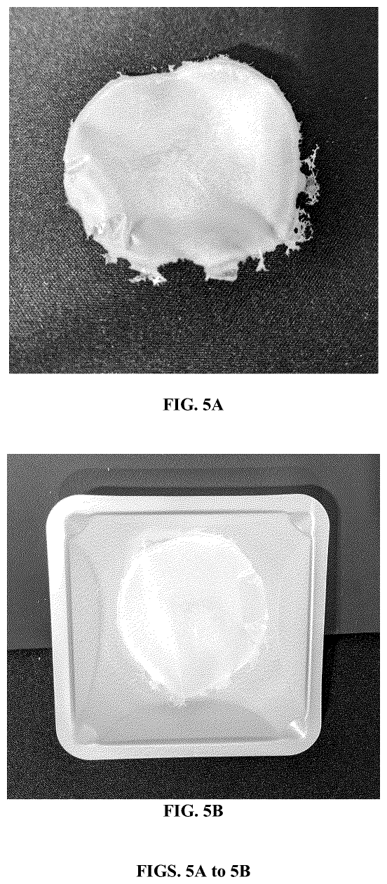

Solutions A and B containing 5 percent PVP by weight were prepared according to Table 12 to form gel A and gel B. About 1 gram (about six pumps from a spray bottle) of gel A and gel B were combined. The amount of PVP was enough to form a skin (see FIGS. 5A and 5B) and create a gel. The addition of ethanol raised the vapor pressure of the solutions to increase the evaporation rate and create a skin more quickly.

TABLE 12

| | Mass/Mass Percent |
|---|---|
| | Based on 100 Percent |
| Gel Solution B | |
| Acid | 1 to 18 |
| PVP | 1 to 30 |
| DI Water | Remainder |
| Ethanol | 5 to 95 |
| Aerosol Solution A | |
| Sodium Nitrite | 1 to 30 |
| PVP | 1 to 30 |
| DI Water | Remainder |
| Ethanol | 5 to 95 |

### 1. No Production

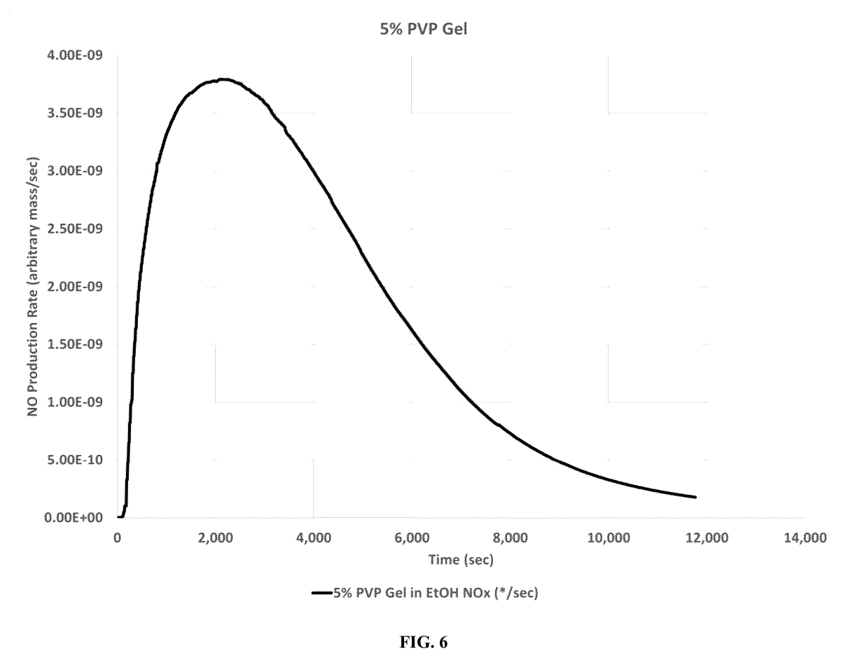

The NO production rate was measured as described previously with the temperature of the pad set to 37° C. The NO production profile for the gel formulation is shown in FIG. 6. The vertical axis of the graph is in arbitrary mass units per second.

### Analysis

The three tests show that foam, gel, and aerosol formulations were created that produced NO that began thickening almost immediately and that created a skin over time (confirmed after about 18-24 hours of drying).

The present invention may be embodied in other specific forms without departing from its fundamental functions or essential characteristics. The described embodiments are to be considered in all respects only as illustrative, and not restrictive. All changes which come within the meaning and range of equivalency of the illustrative embodiments are to be embraced within their scope.

1. A topical nitric oxide delivery system; comprising:
a first topical pharmaceutical solution comprising a nitrite, a liquid selected from the group consisting of water, one or more polar protic liquids, or a mixture of water and one or more polar liquids, and optionally one or both of a surfactant and a dermatological agent;
a second pharmaceutical solution comprising a protic acid and a liquid which is selected form the group consisting of water, one or more polar protic liquids, or a mixture of water and one or more polar protic liquids and optionally one or both of a surfactant and a dermatologic agent; wherein: a surfactant is present in at least one or both of the first and second topical pharmaceutical solution; and wherein:
at least one or both of the first and second topical pharmaceutical solutions comprise a pharmaceutically acceptable excipient or mixture of pharmaceutically acceptable excipients capable of forming a film or gelatinous layer that is comprised of stratified layers of nano-microbubbles containing nitric oxide upon mixing.

2. The topical nitric oxide delivery system of claim 1, wherein the first pharmaceutical solution comprises a pharmaceutically acceptable excipient or mixture of excipients capable of forming a film or gelatinous layer.

3. The topical nitric oxide delivery system of claim 1, wherein the second pharmaceutical solution comprises a pharmaceutically acceptable excipient or mixture of excipients capable of forming a film or gelatinous layer.

4. The topical nitric oxide delivery system of claim 1, wherein the first pharmaceutical solution and the second pharmaceutical solution each comprise the same or different pharmaceutically acceptable excipient or mixture of excipients capable of forming a film or gelatinous layer.

5. The topical nitric oxide delivery system of claims 1-4, wherein:
the first topical pharmaceutical solution is in water or water-alcohol and comprises a nitric oxide donor selected from the group consisting of sodium nitrite and potassium nitrite, or a mixture thereof, and a surfactant selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride; or combinations thereof;
the second topical pharmaceutical solution is in water or water-alcohol and comprises an acid selected from the group consisting of citric acid, lactic acid, and ascorbic acid, or a mixture thereof and a surfactant selected from the group consisting of cetyl trimethyl ammonium bromide, cetrimonium bromide, dodecylbenzenesulfonic acid, cetylpyridinium chloride, stearalkonium chloride, polyquaternium-7, cocamidopropyl betaine, coco betaine, lauryl dimethyl ammonium chloride, polyquaternium-10, behentrimonium chloride, and cetrimonium chloride; or combinations thereof; and
the pharmaceutically acceptable excipient that is capable of forming a film or gelatinous layer is selected from the group consisting of a cellulose, an alginate, a xanthan (gum), a polyols, a polysiloxane, a polyurethane, a carbomer, and polyvinyl pyrrolidone, or mixtures thereof;
wherein the pharmaceutically acceptable excipient that is capable of forming a film or gelatinous layer is present in at least one or both of the first and second pharmaceutical solutions.

6. The topical nitric oxide delivery system of claims 1-5, wherein:
the first topical pharmaceutical solution is in water or water-alcohol and comprises sodium nitrite and coco betaine;
the second topical pharmaceutical solution is in water or water-alcohol and comprises citric acid and coco betaine; and
PVP; wherein the PVP is present in at least one or both of the first and second pharmaceutical solutions.

7. The topical nitric oxide delivery system of claims 1-6, wherein the liquid is water or water-mixed with alcohol, wherein the alcohol is selected from the group consisting of ethanol and isopropanol.

8. The topical nitric oxide delivery system of claims 1-6, wherein the liquid is water-ethanol.

9. The topical nitric oxide delivery system of claims 1-8, further comprising an additional topical pharmaceutical agent.

10. A method for providing nitric oxide therapy to the skin of a human or animal requiring treatment, comprising:
providing a first topical pharmaceutical solution comprising a nitrite, a liquid selected from the group consisting of water, one or more polar protic liquids, or a mixture of water and one or more polar liquids, and optionally one or both of a surfactant and a dermatological agent;
providing a second pharmaceutical solution comprising a protic acid and a liquid which is selected form the group consisting of water, one or more polar protic liquids, or a mixture of water and one or more polar protic liquids and optionally one or both of a surfactant and a dermatologic agent; wherein:
a surfactant is present in at least one or both of the first and second topical pharmaceutical solution;
dispensing the first and second topical pharmaceutical solutions;
mixing the first and second topical pharmaceutical formulations to initiate the production of nitric oxide that is captured in micro- to nano-sized bubbles;
applying the mixture to the skin of the human or animal requiring treatment; and
allowing the mixture to form a film or gelatinous layer comprised of stratified layers of nano- to micro-bubbles containing nitric oxide barrier enclosing the skin of the human or animal requiring treatment and trapping at least a portion of the foam between the barrier and the wound.

11. A method of treating a skin condition of a human or animal, comprising:
combining the first and second topical pharmaceutical formulations recited in claims 1-9 to generate a topical composition comprising nitric oxide, wherein the topical composition is a foam gel, or aerosol;
applying the combination to the skin that requires treatment; and
allowing the mixture to form a barrier enclosing the skin that requires treatment and trapping at least a portion of the topical composition between the barrier and the wound.

12. The method of claim 11, wherein the topical composition is a foam.

13. The method of claim 11, wherein the topical composition is a gel.

14. The method of claim 11, wherein the topical composition is an aerosol.

15. A method of treating a skin condition of a human or animal, comprising
combining the first and second topical pharmaceutical formulations recited in claims 1-9 to generate nitric oxide on the skin requiring treatment to form a mixture, wherein the mixture is a foam, a gel, or an aerosol; and
allowing the mixture to form a barrier enclosing the required treatment and trapping at least a portion of the nitric oxide between the barrier and the wound.

16. The method of claim 15, wherein the mixture is a foam.

17. The method of claim 15, wherein the mixture is a gel.

18. The method of claim 15, wherein the mixture is an aerosol.

19. The method of claims 11-15, wherein the barrier also traps moisture to help with healing.

20. The method of claims 1-15, wherein the barrier protects the skin from abrasions, scrapes, and air- or water-borne pathogens.
